(19) United States
(12) Patent Application Publication
(10) Pub. No.: US 2006/0064046 A1
(43) Pub. Date: Mar. 23, 2006

(54) Cold pack finger splint

(75) Inventors: Marnie A. Kortuem, Tracy, MN (US); Ivan E. Brown, Spirit Lake, IA (US); Teryle L. Kounkel, Spirit Lake, IA (US); Rebecca A. Mills, Spirit Lake, IA (US); Shanna Marks, Spirit Lake, IA (US)

Correspondence Address: MCKEE, VOORHEES & SEASE, P.L.C., 801 GRAND AVENUE, SUITE 3200, DES MOINES, IA, 50309-2721, US

(73) Assignee: BROWN MEDICAL INDUSTRIES, Spirit Lake, IA (US)

(21) Appl. No.: 11/257,941

(22) Filed: Oct. 25, 2005

Related U.S. Application Data
(63) Continuation-in-part of application No. 10/341,557, filed on Jan. 13, 2003.

(51) Int. Cl. A61F 5/00

(52) U.S. Cl. 602/22; 602/5; 602/2; 602/14

(57) ABSTRACT

A finger splint, ice pack gel combination, using a finger splint with an associated ice gel pack so that both support and cold therapy can simultaneously be applied to an injured finger.

### CROSS-REFERENCE TO RELATED APPLICATIONS

This application is a continuation-in-part of U.S. patent application Ser. No. 10/341,557 filed on Jan. 13, 2003, which is a nonprovisional U.S. application.

### FIELD OF THE INVENTION

The present invention relates generally to finger splints and more specifically to a finger splint that can simultaneously provide support for an injured finger and cold therapy to aid in relief from pain and swelling.

### BACKGROUND OF THE INVENTION

Finger splints to protect injured fingers have, of course, been known for years. Typically, such finger splints can be foam splints of the wrap around type, fold-over type, prong splints, or gutter splints. They can be made from metal or plastic.

Constructional features of these conventional finger splints are well known. However, they have application problems. In particular, they are designed for finger protection and support in an immobile position, without necessarily providing comfort or assistance in reducing swelling and pain. As well, such conventional finger splints are often cumbersome, uncomfortable to wear and thus discourage patient compliance.

There is therefore a need for a finger splint that not only protects and immobilizes but also assists in reduction of pain and swelling, and yet comfortable to wear.

It is accordingly an object of the present invention to provide an improved finger splint which provides protection and finger immobilization along with cold therapy for reduction of swelling and pain. The result is quicker healing to allow the injured person to more immediately return to their normal activities of work or play.

Another object of the present invention is to provide an ice pack sheathed splint that will overcome the shortcomings of the prior art finger splints.

A further object of the present invention is to provide an ice pack in conjunction with a splint for providing instant relief from painful finger injuries.

Yet another object is to provide a finger splint ice pack configuration that will serve a dual purpose for injured digits by providing support and protection, as well as a cooling component to minimize pain and swelling.

Another object is to provide a finger splint that will be an affordable way for coaches/clinicians to stabilize an injury until proper medical treatment can be sought.

Another object is to provide a finger splint that any individual without medical skill will be able to apply and adjust with ease.

A still further object is to provide a finger splint that provides a custom fit for fingers of different shapes and sizes due to its flexible, cushioning gel sheath that enshrouds the stiffening member.

Another object is to provide a finger splint wherein 1 or 2 splints may be applied to the same finger to immobilize and simultaneously provide cold therapy to minimize pain and swelling.

A still further object is to provide a finger splint using a flexible internal stiffening member formable to fit the bend of a finger.

Another object is to provide a finger splint wherein the stiffening member is sufficiently rigid to hold form and provide both support and stability to the finger in the bent position.

Another object is to provide a finger splint that will stand up to uses in many different environments without breaking.

Another object is to provide a finger splint that can be secured to the finger using various medical tapes, wrappings and bandages.

Other objects and advantages of the present invention will become apparent from the written description and it is intended that these objects and advantages are all within the scope of the present invention.

To accomplish the above and related objects, this invention may be embodied in the form illustrated in the accompanying drawings; however, the drawings are illustrative only, and changes may be made in the specific constructions illustrated which are preferred embodiments only.

### SUMMARY OF THE INVENTION

In its broadest sense, the invention comprises a finger splint/ice gel pack combination. The combination employs a finger splint of any of the conventional types having an associated ice gel pack so that both support and protection, and ice therapy for pain and swelling, can simultaneously be applied to an injured finger. The invention is specifically contoured to directly target an injured digit by providing ice therapy to the injury through the ice gel pack and support by way of the internal stiffening member. The invention also relates to specific constructional features embodying this concept in a manner that is both economical and will aid in patient compliance. Such constructional details are described in the preferred embodiment set forth below.

### BRIEF DESCRIPTION OF THE DRAWINGS

FIG. 1 is a perspective view of the finger splint encased in a gel wrap or ice pack.

FIG. 2 is a side view of the finger splint encased in a gel wrap or ice pack.

FIG. 3 is a top view of the finger splint encased in a gel wrap or ice pack.

FIG. 4 is an end view of the device of FIG. 1, along lines 2-2.

FIG. 5 is a side view of the device of FIG. 1, along lines 3-3.

FIG. 6 is an in-use view of the finger splint of FIG. 1, wrapped around the fingertip of the injured finger.

FIG. 7 is an in-use view of the finger splint of FIG. 1, bent to conform to the injured finger.

FIG. 8 is an in-use view of the splint of FIG. 1, one splint enveloping the top half of the injured finger and a second splint enveloping the bottom half of the injured finger.

FIG. 9 is an in-use view of a the splint in FIG. 1 enveloping and supporting the bottom half of the injured finger.

### DETAILED DESCRIPTION OF A PREFERRED EMBODIMENT

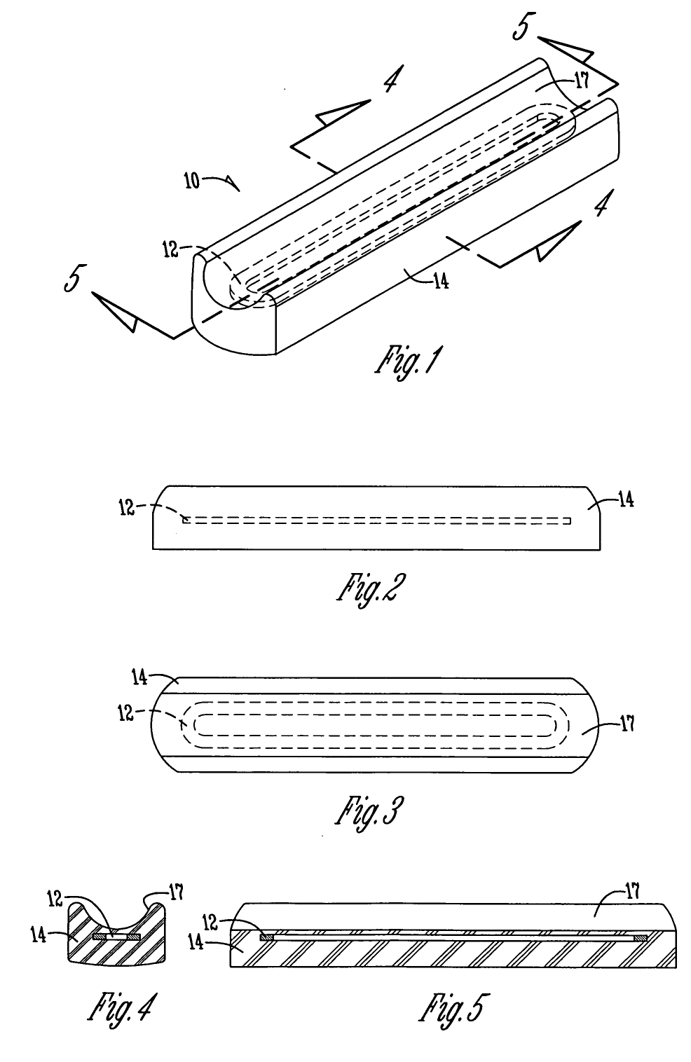

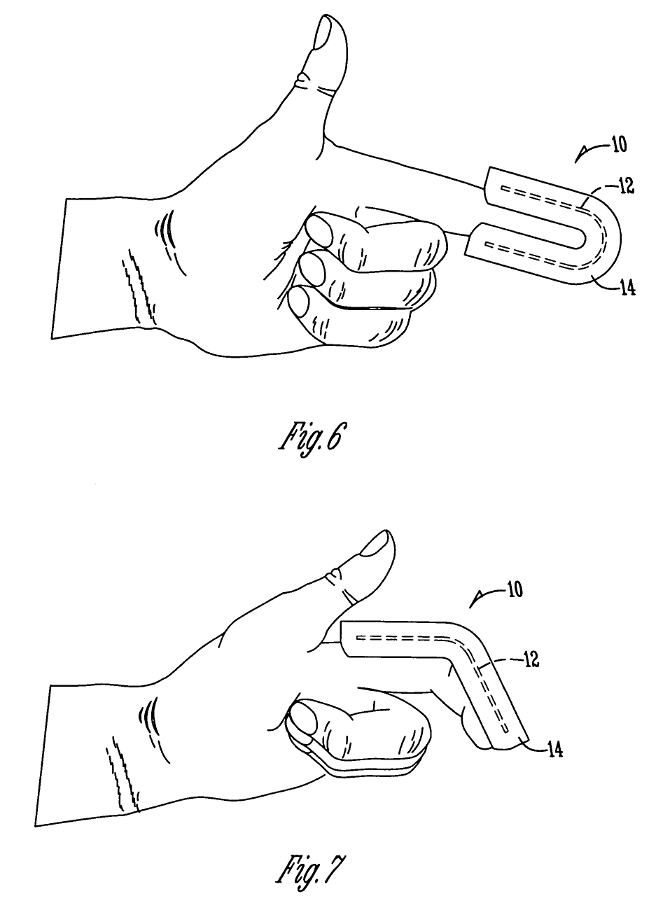

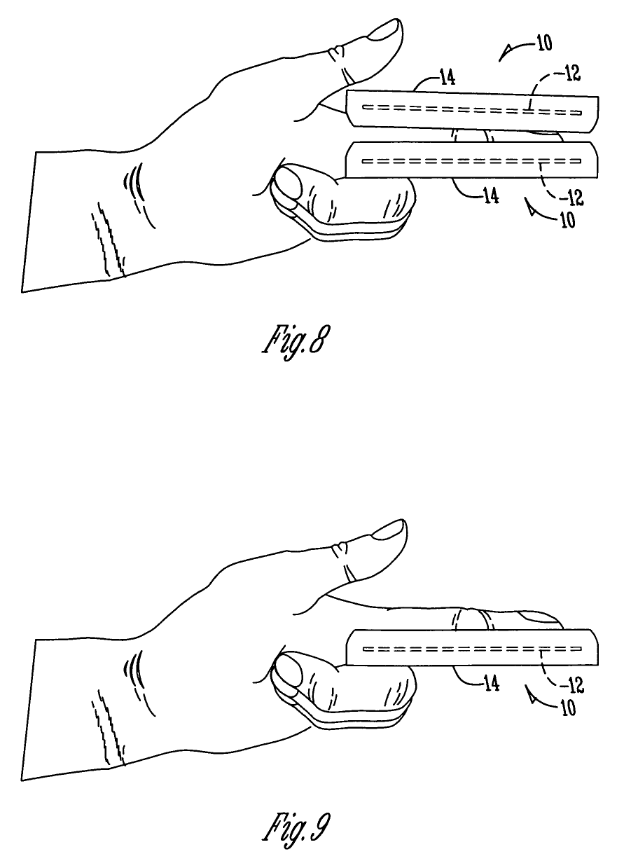

Turning now to the drawings in which similar reference characters denote similar elements through the several views, illustrated in FIGS. 1-9 is the combination of various views and in-use configurations of the finger splint such as folding over the fingertip (FIG. 6), bending to conform to a bent finger (FIG. 7), enveloping the finger between two splints (FIG. 8), supporting a finger with one splint (FIG. 9), and the finger splint shown in detail in FIGS. 1-5. The finger splint being described with particularity herein.

The finger splint ice pack/gel combination, referred to generally as 10 can be used as a fold-over splint (FIG. 6), a conforming gutter splint (FIG. 7), a wrap-around splint (FIG. 8) and standard gutter splint (FIG. 9). Each of these splint designs are available in the art and each are preferred by some users. They all serve the purpose of protection and immobilization.

The finger splint has a stiffening member 12 encased, enshrouded or embedded in a sheath of surrounding gel material 14 (see FIG. 1). The sheath of gel material 14 is an inherently adhering plastisol material. The gel material 14 is also compliant with a spongy feel. The gel material 14 forms the body of the finger splint 10. The body of the finger splint has a contacting surface 17 specially contoured to fit the shape of a finger. The compliant nature of the gel material 14 and stiffening member 12 ensures that the contacting surface 17 fits snugly and forms to the finger. The stiffening member 12 also allows the finger splint 10 be bent, retain a desired shape and provide support and cold therapy to the finger. The finger splint 10 is sufficiently compliant to conform to the finger in a bent position (see FIG. 7). The body of gel material 14 not only helps to provide support and protection for the injured finger, but also retains and provides cold energy to the contacting surface 17 during use. The gel material 14 is commercially available. One suitable gel material 14 is sold by Three G Corporation of 110 West 3^{rd} North Street, Morristown, Tenn. 37814. Such gel material need not be described with chemical particularity except to say that such materials are Plastisols and present a soft cushioning feel, even when subjected to freezing temperatures. They maintain cold temperatures when exposed to ambient 20° C. temperatures for at least 30 minutes, and in some cases, 60 minutes. Other therapeutic gel materials may be employed, for example the gel material referenced in commonly owned U.S. Pat. No. 6,267,742 relating to a night splint suspension system that can be used in combination with Sealed Ice™. Material safety data sheets are available from Three G Corporation for the Plastisol compound suitable for use in the present invention.

One of the advantages of the gel material 14 is that it may be used unchilled to provide soft, cushioned extraordinary comfort; or, it can be chilled to provide sustained cold therapy for pain and swelling.

The splint 10 is manufactured in the following way. The stiffening member 12 is suspended in a mold. Melted therapy gel is poured into the mold shaped to form the body of the finger splint 10, including the contacting surface 17. The gel is allowed to cool and the splint 10 with the encased stiffening member 12 is removed from the mold.

In use, the splint 10 is removed from its package, usually cold, and then placed over and bent into position to conform to the injured finger. The splint 10 provides support and protection and the gel 14 supplies cold therapy, comfort and cushioning

As used herein, the terms “cold pack”, or “ice gel pack” or “ice pack”, refer to the temperature of the gel pack and not its composition. Put another way, the material is not referred to as “ice” because of water content but because of its temperature, i.e. ice cold.

The finger splint 10 can conform to various angles of bend in the injured finger and adapt to the various needs of the user. FIGS. 6-9 illustrate, in part, the flexibility of the finger splint 10. FIG. 6 shows the splint 10 bent over the fingertip to provide support, protection and cold therapy. FIG. 7 depicts the splint 10 conforming to the shape of the injured finger in a bent position to provide support, protection and cold therapy. FIG. 8 illustrates the injured finger encapsulated by two splints 10 to apply support, protection and cold therapy to the whole finger. Lastly, FIG. 9 shows the splint 10 being used as a standard gutter splint, providing support, protection and cold therapy to underside of the injured finger. It is preferred that the finger splint 10 in FIGS. 6-9 be secured to the finger using various medical tapes, wrappings and bandages.

The invention has been shown and described above with the preferred embodiments, and it is understood that many modifications, substitutions, and additions may be made which are within the intended spirit and scope of the invention. From the foregoing, it can be seen that the present invention accomplishes at least all of its stated objectives.

What is claimed is:

1. A cold therapy finger splint combination comprising:
a finger splint having a stiffening member enveloped by a gel pack so that both support and cold therapy can simultaneously be applied to a finger in need of such treatment.

2. The finger splint of claim 1 wherein the gel pack further comprises a contacting surface having a contour to fit the finger.

3. The finger splint of claim 2 wherein the surface and the contour are inherently adhering.

4. The finger splint of claim 3 wherein the cold therapy is applied across the contacting surface.

5. The finger splint of claim 4 wherein the cold therapy is supplied to the contacting surface by the gel pack.

6. The finger splint of claim 1 wherein the gel pack and the contacting surface are a polymeric plastic.

7. The finger splint of claim 1 wherein the stiffening member is flexible.

8. The finger splint of claim 1 wherein the stiffening member is aluminum.

9. A finger splint gel pack combination, comprising:
a finger splint sheathed in a gel ice pack.

10. The finger splint of claim 9 wherein the finger splint is fully enclosed by the gel ice pack.

11. The finger splint of claim 10 wherein at least one finger splint is used to apply support and cold therapy to a finger.

12. The finger splint of claim 11 wherein the finger splint is secured to the finger by folding over the fingertip.

13. A finger splint, comprising:
a finger splint encased in a body having a contacting surface;
said body being an ice pack to provide cold therapy across the contacting surface while said finger splint provides finger support.

14. A method using a finger splint, comprising:
shaping the splint encased within an ice-gel pack to fit a finger;
fitting the finger to the ice-gel pack having a contacting surface;
seating the contacting surface to the finger so that both support and cold therapy can simultaneously be applied to the finger in need of such treatment.
